(19) United States
(12) Patent Application Publication
(10) Pub. No.: US 2003/0004214 A1
(43) Pub. Date: Jan. 2, 2003

(54) Use of rofleponide in the treatment of irritable bowel syndrome (ibs)

(76) Inventor: Ralph Brattsand, Lund (SE)

Correspondence Address: WHITE & CASE LLP, PATENT DEPARTMENT, 1155 AVENUE OF THE AMERICAS, NEW YORK, NY, 10036, US

(21) Appl. No.: 10/181,345

(22) PCT Filed: Jan. 15, 2001
(86) PCT No.: PCT/SE01/00069

(30) Foreign Application Priority Data
Jan. 31, 2000 (SE) 0000332-7

(51) Int. Cl. A61K 31/23

(52) U.S. Cl. 514/552

(57) ABSTRACT

The invention provides the use of rofleponide, its esters and salts in the manufacture of a medicament for use in the treatment of irritable bowel syndrome (IBS) and pharmaceutical formulations for use in such treatment.

### FIELD OF THE INVENTION

The present invention provides a new treatment for irritable bowel syndrome (IBS), namely use of rofleponide, its esters and salts.

### BACKGROUND OF THE INVENTION

The irritable bowel syndrome is a chronic abdominal disease for which there is no apparent underlying structural cause. Symptoms in IBS are thought to arise from altered gastro-intestinal motility, increased visceral sensitivity or altered brain-gut modulation. The diagnosis of IBS is hampered by the absence of simple diagnostic tests. Physicians approach IBS as a diagnosis of exclusion and then base the diagnosis on certain diagnostic criteria such as abnormal discomfort and pain, bloating and disturbed defecation, see further in Gut, 1999; 45 (Suppl.2):II43, C1(Sept), Thompson et al., and Gasteroenterology 1997, vol. 112, p.2120-2137. Current treatment of IBS is mainly antispasmodics, laxatives, loperamide and antidepressants. A history of gastro-enteritis (Salmonella, Campylobacter etc.) is more commonly found in patients with IBS than in a control population and up to 30% of patients develop IBS after gastro-enteritis.

### SUMMARY OF THE INVENTION

According to the invention there is provided the use of rofleponide, its esters and salts, such as fatty acid esters e.g. rofleponide palmitate in the manufacture of a medicament for use in the treatment of irritable bowel syndrome, particularly post-infectious irritable bowel syndrome.

According to the invention there is further provided a method of treating a patient suffering from irritable bowel syndrome which comprises administering to the patient a therapeutically effective amount of rofleponide, its esters and salts, such as fatty acid esters e.g. rofleponide palmitate.

According to the invention there is further provided a pharmaceutical formulation for use in the treatment of irritable bowel syndrome wherein the active ingredient is rofleponide, its esters and salts, such as fatty acid esters e.g. rofleponide palmitate.

It has now surprisingly been found that the rofleponide substance used in the present invention which has a minimal systemic effect and has a first pass metabolism of at least 99% is effective in the treatment of irritable bowel syndrome (IBS). Compared to other very potent topical steroids rofleponide has i) unique combination of a sufficient water solubility for dissolution distribution in intestinal fluids, ii) a very high affinity for and activity at glucocorticosteroid receptors, and iii) a nearly complete first pass inactivation by cytochrome P450 enzymes in the intestinal hepatic region, giving an oral bioavailability of ≦1%.

Rofleponide is chemically named (22R)-16-alpha, 17 alpha-butylidenedioxy-6-alpha, 9alpha-difluoro-11beta,21-dihydroxypregn-4-ene-3,20-dione.

When rofleponide, its esters and salts is administered orally, it is administered oesophageally, generally administered in the form of tablets, pills, capsules, syrups, powders or granules and when it is administered rectally, is in the form of suppositories or enemas.

Rofleponide, its esters and salts may be administered on its own or as a pharmaceutical formulation in combination with a pharmaceutically acceptable diluent, adjuvant or carrier. Particularly preferred are compositions not containing material capable of causing an adverse, e.g. an allergic reaction.

Rofleponide, its esters and salts may be admixed with an adjuvant or a carrier. e.g. lactose, saccharose, sorbitol, mannitol, starches such as potato starch, corn starch or amylopectin, cellulose derivatives, in an organic salts such as calcium sulphates, a binder such as gelatine or polyvinylpyrrolidone, and a lubricant such as magnesium stearate. calcium stearate, polyethylene glycol, waxes, paraffin, and the like, and then compressed into tablets. If coated tablets are required, the cores, prepared as described above. may be coated with a concentrated sugar solution which may contain e.g. gum arabic. gelatine, talcum, titanium dioxide, and the like. Alternatively, the tablet may be coated with a suitable polymer dissolved in a suitable organic solvent or with a polymer dispersion in water. Suitable polymers include cellulose derivatives, plyvinylpyrrolidone or acrylates. The tablet, capsule or granules, preferably has an enteric coating to allow release of the drug in the intestine, particularly the lower intestine. Suitable capsules may be prepared by using the methods described in EP-A-502092, WO 95/08323 or WO 97/27843.

For the preparation of soft gelatine capsules, the rofleponide, its esters and salts may be admixed with e.g. a vegetable oil or polyethylene glycol. Hard gelatine capsules may contain granules of the compound using the above mentioned excipients. Also liquid or semisolid formulations of the drug may be filled into hard gelatine capsules.

Liquid preparations for oral application may be in the form of syrups or suspensions, for example solutions containing the active compound, the balance being sugar and/or a mixture of ethanol, water, glycerol and propylene glycol. Optionally such liquid preparations may contain colouring agents, flavouring agents, saccharine and carboxy-methylcellulose as a thickening agent or other excipients known to those skilled in the art.

Rofleponide, its esters and salts is preferably administered at a dosage of from 0.1 to 20 mg, more preferably from 0.5 to 10 mg, either as a single dose or in divided doses from 2 to 4 times per day.

For testing the effect of rofleponide, its esters and salts in post-infectious irritable bowel syndrome the intestinal neuromuscular dysfunction after acute nematode infection in mice are measured in accordance with the method described in Gasteroenterology 1997, vol. 133, p.1224-1232.

1. Use of rofleponide, its esters and salts in the manufacture of a medicament for use in the treatment of irritable bowel syndrome.

2. Use according to claim 1 wherein the medicament is administrated orally or rectally.

3. Use according to any one of claims 1-2 wherein the medicament comprises rofleponide, its esters and salts in an amount, which provides a daily dose of from 0.1 to 20 mg.

4. Use according to claim 3 wherein the medicament is administered as a single daily dose or in from 2 to 4 divided doses.

5. Use according to any one of claims 1-4 wherein the rofleponide ester is a fatty acid ester.

6. Use according to claim 5 wherein the rofleponide fatty acid ester is rofleponide palmitate.

7. A method of treating a patient suffering from irritable bowel syndrome which comprises administering to the patient a therapeutically effective amount of rofleponide, its esters and salts.

8. The method according to claim 7 which comprises administering orally or rectally of rofleponide, its esters and salts.

9. The method according to claims 7 or 8 which comprises administering rofleponide, its esters and salts in an amount, which provides a daily dose of from 0.1 to 20 mg.

10. The method according to claim 9 wherein rofleponide, its esters and salts is administered in a single daily dose or in from 2 to 4 divided doses.

11. The method according to any one of claims 7-10 wherein the rofleponide ester is a rofleponide fatty acid ester.

12. The method according to claim 11 wherein the rofleponide fatty acid ester is rofleponide palmitate.

13. A pharmaceutical formulation for use in the treatment of irritable bowel syndrome wherein the active ingredient is rofleponide, its esters and salts.

14. The pharmaceutical formulation according to claim 13, which is administrated orally or rectally.

15. The pharmaceutical formulation according to any one of claims 13-14 comprises rofleponide, its esters and salts in an amount which provides a daily dose of from 1 to 20 mg.

16. The pharmaceutical formulation according to claim 15 wherein rofleponide, its esters and salts is administered as a single daily dose or in from 2 to 4 divided doses.

17. The pharmaceutical formulation according to any one of claims 13-16 wherein the rofleponide ester is a fatty acid ester.

18. The pharmaceutical formulation according to claim 17 wherein the rofleponide fatty acid ester is rofleponide palmitate.
